(19) United States
(12) Patent Application Publication
(10) Pub. No.: US 2005/0170348 A1
(43) Pub. Date: Aug. 4, 2005

(54) Nuclear-envelope and nuclear-lamina binding chimeras for modulating gene expression

(76) Inventor: Takashi Sera, Kyoto (JP)

Correspondence Address: JENKINS, WILSON & TAYLOR, P. A., 3100 TOWER BLVD, SUITE 1400, DURHAM, NC, 27707, US

(21) Appl. No.: 10/500,671

(22) PCT Filed: Jan. 17, 2003
(86) PCT No.: PCT/US03/01529

Related U.S. Application Data
(60) Provisional application No. 60/350,163, filed on Jan. 18, 2002.
(60) Provisional application No. 60/351,315, filed on Jan. 23, 2002.

(51) Int. Cl. C12Q 1/68; C07H 21/04; C12N 9/22

(52) U.S. Cl. 435/6; 435/69.1; 435/199; 435/320.1; 435/325; 536/23.2

(57) ABSTRACT

The present invention is directed to nucleic acid target-specific chimeric proteins comprising a nuclear-envelope and/or nuclear-lamina binding domain and a DNA binding domain. These proteins, as well as the nucleic acids encoding those proteins, can be used in methods to repress or down-regulate expression of selected genes. The DNA binding domains are preferably from naturally-occurring zinc finger proteins (ZFPs) or artificial zinc finger proteins (AZPs). Molecular switch systems for gene regulation are also provided.

### FIELD OF THE INVENTION

The present invention is directed to nucleic acid target-specific chimeric proteins comprising a nuclear-envelope and/or nuclear-lamina binding domain and a DNA binding domain. These proteins, as well as the nucleic acids encoding those proteins, can be used in methods to modulate gene expression and are particularly useful to repress or down-regulate expression of selected target genes. The DNA binding domains are preferably from naturally-occurring zinc finger proteins (ZFPs) or artificial zinc finger proteins (AZPs). The invention also relates to molecular switch systems for gene repression and derepression.

### BACKGROUND OF THE INVENTION

Transcriptional repression of genes can be achieved by a variety of mechanisms. A classic example is the lac repressor which, when bound to its target sequence on the lac operon, prevents RNA polymerase from binding and thereby initiating transcription. In eukaryotes, additional mechanisms exist to control gene repression. For example, genes found in constitutive heterochromatin are transcriptionally silent. Heterochromatin is not positioned randomly and appears to be associated with the nuclear periphery [Cohen et al. (2001) Trends Biochem. Sci. 26:41-47], suggesting that bringing genes into proximity with heterochromatin or the nuclear periphery may play a role, at least in part, in gene silencing.

Transcriptional repressors are also found at the nuclear periphery in eukaryotes. In some cases, it appears that such proteins are only active as repressors when localized to the nuclear periphery. The nuclear periphery of higher eukaryotes (metazoans and above) consists of a nuclear envelope (NE) with inner and outer membranes and a nuclear lamina. The nuclear lamina resides underneath the inner nuclear membrane and is composed of intermediate filaments termed lamins and lamina-associated proteins (LAPs). Certain LAPs are also integral membrane proteins of the inner nuclear membrane. A discussion of the composition of the nuclear lamina from several different species is provided in Cohen et al.

Oct-1 is a repressor of the aging-associated collagenase gene. Experimental evidence shows that dissociation of Oct-1 from the nuclear periphery induces collagenase gene expression [Imai et al. (1997) Mol. Biol. Cell 8:2404-2419]. Furthermore, when the active form of the retinoblastoma protein (Rb) is associated with the transcription factor E2F, the complex co-localizes with lamins A/C at the nuclear periphery in vivo and represses transcription [Mancini et al. (1999) Dev. Biol. 215:288-297]. The mouse germ-cell-less protein (GCL), also involved in gene repression, [Nihi et al. (2001) J. Cell. Sci. 114:3297-3307], has been reported to bind LAP2∃ at the nuclear lamina (Cohen et al.).

Transcription factors and other DNA binding proteins bind their targets in a sequence specific manner to modulate gene expression and thereby activate or repress expression of the target gene. Modulation of gene expression can be achieved temporally (e.g., at different times in development or during the cell cycle) and/or spatially (e.g., in different tissues). In some instances, it may be desirable to turn off expression of undesired genes at particular times or in particular cell types. For example, genes that become associated with and activated in oncogenesis may be targets for repression. Since heterochromatin and genes localized to the nuclear periphery are known to be silenced, a sequence-specific method to bring a gene into association with the nuclear periphery could provide a route to silence or down regulate (repress) expression of that target gene. Alternatively, a method to release genes from a state of repression (i.e., to derepress or activate those genes) would also be valuable.

However, known transcription factors have limited utility—such proteins are useful to control genes associated with their natural target sequences or to a limited set of closely related target sequences. One way to overcome this drawback is to design and construct DNA binding proteins with predetermined sequence specificity, particularly for unique target sequences in a large, complex genome. One particular class of proteins shown amenable to such manipulation is zinc finger proteins (ZFPs)

ZFPs are well-known DNA-binding proteins that recognize and bind to DNA target sequences by interaction of the target sequence with particular amino acids in the alpha helix of each zinc finger in the ZFP. ZFPs typically contain from three to nine, and sometimes more, zinc fingers and there are many classes of ZFPs; for a review, see, e.g., Laity et al. (2001) Curr. Opin. Struct. Biol. 11:3946. The Cys₂His₂ class of ZFPs has been extensively studied and proved particularly useful in development of a universal recognition code to permit the design of artificial zinc finger proteins (AZPs) that bind predetermined DNA target sequences. See, e.g., Wolfe et al. (2000) Ann. Rev. Biophys. Biomol. Struct. 29:183-212; Choo et al. (2000) Curr. Opin. Struct. Biol. 10:411-416; Segal et al. (1999) Proc. Natl. Acad. Sci. USA 96:2758-2763; Kim et al. (1998) Proc. Natl. Acad. Sci. USA 95:2812-2817; and U.S. Ser. No.09/911,261 to Takashi Sera, filed Jul. 23, 2001 and entitled “Zinc Finger Domain Recognition Code and Uses Thereof.”

The availability of AZPs enables the design of proteins that can regulate target genes associated with any unique sequence not just known regulatory sequences. When these AZPs (or other DNA binding proteins) are combined with one or more protein domains capable of associating with the nuclear periphery, a chimeric protein is created which can be used to bind a nucleotide sequence associated with a target gene and localize that target gene to the nuclear periphery for silencing or down regulation. When the domains of these chimeric proteins are rearranged into a molecular switch system, it is possible to provide systems for either activation or repression of gene expression.

### SUMMARY OF THE INVENTION

The present invention relates to nucleic acid target-specific, chimeric proteins having one or more first domains capable of specifically binding a nucleotide sequence associated with a target gene and having one or more second domains capable of associating with or binding to the nuclear periphery. These proteins are useful in regulating gene expression. Multiple first and second domains, preferably from one to five additional domains can also be present in the chimeric proteins of the invention. The preferred first domain is an AZP and the preferred second domain is a GCL protein. In certain embodiments the chimeric proteins can include additional domains to facilitate cellular uptake and/or transport to the nucleus.

Other aspects of the invention provide isolated nucleic acids encoding the chimeric proteins of the invention, expression vectors comprising those nucleic acids, and host cells transformed (by any method) with the expression vectors. Such host cells can be used, e.g., in a method of preparing the chimeric protein by culturing the host cell for a time and under conditions to express the chimeric protein and recovering the chimeric protein. In addition the host cells can be used as a source of expression vectors to deliver the chimeric protein by gene transfer methods into a cell or an organism. In addition, the invention provides pharmaceutical compositions of these chimeric proteins, nucleic acids and expression vectors.

A still further aspect of the invention relates to a method of binding a target nucleic acid with chimeric protein of the invention by contacting the target nucleic acid (having a nucleotide sequence associated with the target gene) with a chimeric protein of the invention in an amount and for a time sufficient for that protein to bind to the target nucleic acid. In a preferred embodiment the chimeric protein is introduced into a cell via a nucleic acid for in vivo binding. Alternatively, the method provides the chimeric protein can be used for an in vitro binding assay.

A yet further aspect of the invention provides a method of repressing or down regulating expression of a target gene which comprises contacting a nucleic acid containing a nucleotide sequence associated with or in sufficient proximity to the target gene with a chimeric protein of the invention in an amount and for a time sufficient for the protein to decrease the expression level of the target gene relative to an appropriate control. In certain embodiments, the chimeric protein is introduced into a cell or an organism as a protein or as a nucleic acid encoding the chimeric protein.

In the contemplated method of binding a target nucleic acid or the contemplated methods of repressing gene expression, the target gene encodes, or the targeted nucleotide sequence site is from or controls, a plant gene, a mammalian gene, an insect gene, a yeast gene or is from a virus such as a DNA virus. When the target gene or site is from a mammal, it can encode or control a cytokine, an interleukin, an oncogene, an anti-angiogenesis factor, a drug resistance gene and/or any other desired target which allows a selected gene to be brought into proximity with the nuclear periphery and thereby silenced or down regulated. Plant genes of interest include, but are not limited to, genes from tomato, corn, rice and cereal plants. Moreover, multiple target genes that share a common nucleotide target sequence can be coordinately or simultaneously controlled.

A still further aspect of the invention relates to molecular switch systems useful for gene repression. These systems comprise (a) a first fusion protein with a first domain capable of specifically binding a nucleotide sequence associated with a target gene, and a second domain capable of specifically binding to a first binding moiety of a divalent ligand, where the ligand is capable of uptake by a cell, and the first domain and second domains are heterologous with respect to each other; and (b) a second fusion comprising a first domain capable of associating with the nuclear periphery and a second domain capable of specifically binding to the second binding moiety of the divalent ligand. The first domain of the first fusion protein is the same as the first domain of the chimeric proteins of the invention; and the first domain of the second fusion protein is the same as the second domain of the chimeric proteins of the invention. The second domains of the two fusion proteins can be a single chain variable region (scFv) of an antibody with specificity for its respective binding moiety of the divalent ligand.

Other aspects of the invention provide isolated nucleic acids encoding the fusion proteins for gene repression of the invention, expression vectors comprising those nucleic acids, and host cells transformed (by any method) with the expression vectors. Such host cells can be used in a method of preparing the fusion proteins by culturing the host cell for a time and under conditions to express the fusion proteins and recovering the fusion proteins. In addition the host cells can be used as a source of expression vectors to deliver the fusion proteins by gene transfer methods into a cell or an organism. In addition, the invention provides pharmaceutical compositions of these fusions proteins, the molecular switch systems, nucleic acids and expression vectors.

The molecular switch useful for gene repression can be used in a method of temporally or spatially repressing expression of a target gene by (a) contacting a cell or an organism containing a target nucleic acid having a nucleotide sequence associated with a target gene with these molecular switch systems, and (b) contacting the cell or organism with the divalent ligand of the molecular switch system at a time or in a location to allow formation of a complex between the fusion proteins to thereby repress expression of the said target gene by localizing the target gene to the nuclear periphery. The fusion proteins of this molecular switch system can be introduced into the cell or organism as proteins, as one or more nucleic acids encoding one or more of those proteins, or as a combination thereof.

Yet another aspect of the invention relates to molecular switch systems useful for gene derepression, i.e., activation of repressed genes. These systems comprise (a) a first fusion protein comprising a first domain capable of specifically binding a nucleotide sequence associated with a target gene, and a second domain capable of specifically binding to a binding partner, where the first and second domains are heterologous with respect to each other; and (b) a second fusion protein comprising a first domain capable of associating with the nuclear periphery and a second domain comprising the binding partner of the second domain of said first fusion protein, wherein said first domain is heterologous with respect to said second domain. The first domain of the first fusion protein is the same as the first domain of the chimeric proteins of the invention; and the first domain of the second fusion protein is the same as the second domain of the chimeric proteins of the invention. The second domain of the first fusion protein can be an S-protein and the second domain of said second fusion protein can be an S-tag, or vice-a-versa.

Other aspects of the invention provide isolated nucleic acids encoding these fusion proteins for gene derepression of the invention, expression vectors comprising those nucleic acids, and host cells transformed (by any method) with the expression vectors. Such host cells can be used in a method of preparing the fusion proteins by culturing the host cell for a time and under conditions to express the fusion proteins and recovering the fusion proteins. In addition the host cells can be used as a source of expression vectors to deliver the fusion proteins by gene transfer methods into a cell or an organism. In addition, the invention provides pharmaceutical compositions of these fusions proteins, the molecular switch systems, nucleic acids and expression vectors.

The molecular switch useful for gene derepression can be used in a method of temporally or spatially altering expression of a target gene by (a) contacting a cell or an organism containing a target nucleic acid having a nucleotide sequence associated with a target gene with these molecular switch systems, and (b) contacting the cell or organism with a ligand of the molecular switch system at a time or in a location to disrupt association of the first and second fusion proteins and thereby derepress expression of said target gene. by releasing the target gene from its association with the nuclear periphery. The fusion proteins of this molecular switch system can be introduced into the cell or organism as proteins, as one or more nucleic acids encoding one or more of those proteins, or as a combination thereof.

### BRIEF DESCRIPTION OF THE DRAWINGS

FIG. 1 schematically illustrates monomeric and polymeric gene repression using chimeric proteins of the invention to bring one or more target genes into proximity to the nuclear periphery.

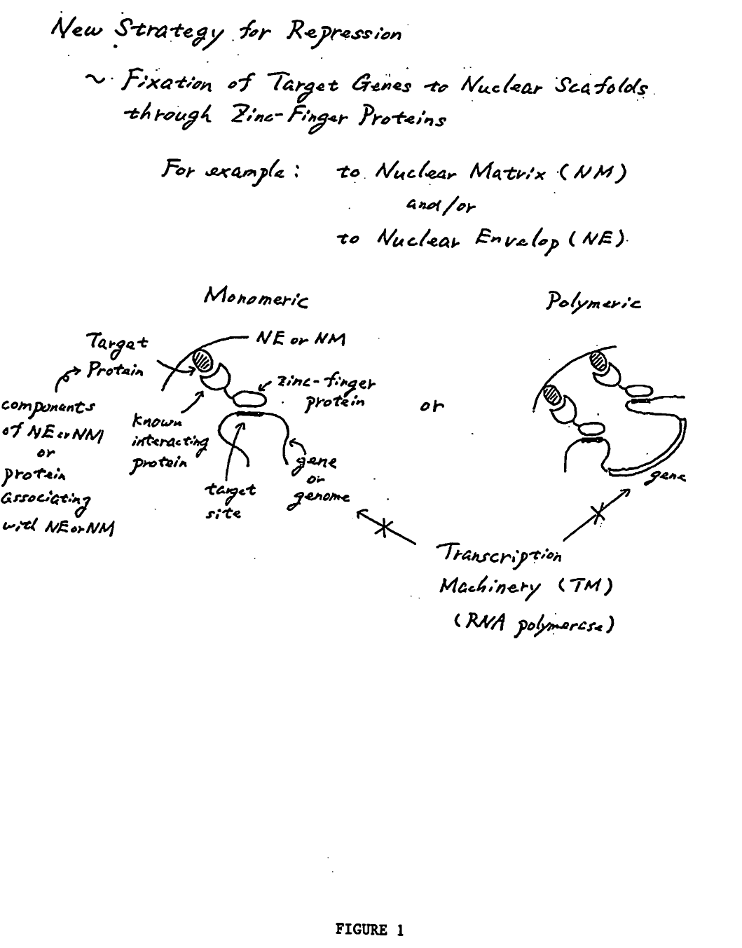

### DETAILED DESCRIPTION OF THE INVENTION

A. Chimeric Proteins of the Invention

The present invention relates to target-specific, chimeric proteins for repressing gene expression by bringing a target gene into proximity to the nuclear periphery and thereby silence or down regulate expression of that gene. The chimeric proteins comprise at least two heterologous domains: a first domain capable of specifically binding a nucleotide sequence associated with the target gene, and a second domain capable of associating with the nuclear periphery by binding to or associating with proteins at or in the nuclear envelope, nuclear lamina, heterochromatin or any combination of the three. The chimeric proteins of the invention are useful in regulating gene expression, particularly to repress or down regulate expression of the selected gene. For example, it may be desirable to down regulate or shut off genes involved in oncogenesis, cellular proliferation and regeneration, angiogenesis (when unwanted blood vessel formation occurs such as in tumors), or in plants at particular stages of development or growth. Similarly, the chimeric proteins of the invention can be used to down regulate or shut off viral genes.

As used herein, the term “nuclear periphery” includes the nuclear envelope and the nuclear lamina. A gene in proximity to the nuclear periphery is physically adjacent to the nuclear periphery and, in accordance with the invention, is so positioned by forming an association (covalently or non-covalently) with proteins that bind to or form part of the nuclear envelope, the nuclear lamina or heterochromatin associated with the nuclear envelope or nuclear lamina. For purposes of the present invention, it is not necessary to determine the actual physical location of a gene relative to the nuclear periphery, but rather, one can measure and use the reduction in gene expression relative to the normal expression level, or other control level of expression, to assess whether the gene is at or in proximity to the nuclear periphery.

As used herein, the term “chimeric protein” or “chimeric proteins” is used to denote that the proteins of the invention are non-naturally occurring proteins. The chimeric proteins of the invention are artificial constructs combining a nucleic acid binding domain and a domain capable of associating with the nuclear periphery from different sources, i.e., the two domains are heterologous with respect to each other. When multiple domains are present, it is sufficient that only one nucleic acid binding domain be from a source different from the domain capable of associating with the nuclear periphery. The sources of the heterologous domains can be, independently, from different species, from different strains of an organism, from different proteins of a single organism or from artificial proteins designed to have the desired activity, provided that none of the combinations are such to produce a naturally-occurring protein.

The nucleic acid binding domain of the chimeric protein specifically binds to a nucleotide sequence associated with the target gene. The identity and characteristics of that domain is determined by the nucleotide sequence desired to be bound by the chimeric protein. As used herein, “specifically binds” means, and includes reference to, the binding or association of a DNA binding moiety or protein (for example, as a whole protein, as a domain, or as present in a chimeric protein of the invention) to a specified nucleotide sequence to a detectably greater degree (e.g., at least 1.5-fold over background) than its binding to other nucleotide sequences and to the substantial exclusion of other nucleotide sequences under a particular set of conditions, such, e.g., as temperature, ionic strength, solvent polarity and the like. The gel shift assay, well known in the art, is one method useful to assess and verify whether the binding is specific for a particular nucleotide sequence.

It is possible to control the nature and position of the nucleotide sequence relative to the target gene. As used herein, a “target polynucleotide,” “nucleotide sequence associated with a target gene,” or “targeted nucleotide sequence,” or other similar terminology refers to a portion of a double-stranded polynucleotide, preferably DNA, to which the DNA binding domain of the chimeric proteins binds. This targeted nucleotide sequence may be at any location, near or within the target gene to be regulated, provided that location is suitable for repressing expression of that target gene. For example, the targeted nucleotide sequence can be within the coding region, immediately upstream or downstream thereof or it can be some distance away (e.g., several hundred nucleotides) if the selected nucleotide sequence still allows the gene to be brought into sufficient proximity to the nuclear periphery to reduce expression of that gene from its normal or other control level. The targeted nucleotide sequence can also be all or part of a known transcriptional control element for a target gene.

The length of the targeted nucleotide sequence can range from about 6-10 nucleotides to about 50, 60, 70 or more nucleotides. Examples of suitable nucleotide sequence lengths are about 8 to about 30, about 10 to about 25, and about 10 to about 20 nucleotides. A length of about 16 nucleotides is sufficient to provide a unique target site in the human genome. The specificity and affinity of the DNA binding domain, the organism being targeted and the nature of the sequence can all be factors in determining the appropriate length of the targeted nucleotide sequence. Those of skill in the art can readily determine the length and identity of the targeted nucleotide sequence based on such considerations.

The nucleic acid binding domain of the chimeric protein can be a known or artificial DNA binding protein or a fragment thereof with DNA binding activity. Examples of DNA binding proteins include, but are not limited to, zinc finger proteins (ZFPs), artificial zinc finger proteins (AZPs), the DNA binding moiety of a transcription factor, nuclear hormone receptors, homeobox domain proteins such as engrailed or antenopedia, helix-turn-helix motif proteins such as lambda repressor and tet repressor, Gal4, TATA binding protein, helix-loop-helix motif proteins such as myc and myoD, leucine zipper type proteins such as fos and jun, and beta-sheet motif proteins such as met, arc, and mnt repressors, or the DNA binding moiety of any of those proteins. Such proteins and moieties are known to those of skill in the art.

The preferred DNA binding proteins for the nucleic acid binding domains of the invention are ZFPs and AZPs. There are many classes of ZFPs, including but not limited to, Cys₂His₂ class (examples, Sp1C and Zif 268), Cys₆ (example, the Gal4 DNA binding protein) and Cys₄ (example, estrogen hormone receptor); any of these proteins with the desired nucleotide sequence specificity can be used.

By “zinc finger protein”, “zinc finger polypeptide,” “ZFP,” “artificial zinc finger protein” or “AZP” is meant a polypeptide having DNA binding domains that are stabilized by zinc. The individual DNA binding domains are typically referred to as “fingers,” such that a ZFP or peptide has at least one finger, more typically two fingers, more preferably three fingers, or even more preferably four or five fingers, to at least six or more fingers. Each finger binds three or four base pairs of DNA. In the Cys₂-His₂ class of ZFPs and AZPs, each finger is typically an approximately 30 amino acid, zinc-chelating, DNA-binding moiety domain. A representative sequence motif for the Cys₂-His₂ class is -Cys-(X)₂₋₄-Cys-(X)₁₂-His-(X)₃₋₅-His, where X is any amino acid (SEQ ID NO: 1). The two invariant histidine residues and the two invariant cysteine residues bind a zinc cation [see, e.g., Berg et al. (1996) Science 271:1081-1085].

In one embodiment of the invention, the chimeric protein has a first domain which is an AZP comprising at least one zinc finger, each finger represented by the formula

| | |
|---|---|
| -X₃-Cys-X₂₋₄-Cys-X₅-Z⁻¹-X-Z²-Z³-X₂-Z⁶-His-X₃₋₅-His-X₄-, | (SEQ ID NO: 2) |

and where multiple fingers, when present, are independently covalently joined to each other with from 0 to 10 amino acid residues, wherein X is any amino acid, Xₙ represents the number of occurrences of X in the polypeptide chain and Z⁻¹, Z², Z³, and Z⁶ are determined by a recognition code shown in Tables 1 and 2 (and as further explained below).

The amino acids represented by X form the framework of a Cys₂His₂ zinc finger and can be a known zinc finger framework, a consensus framework, a framework obtained by varying the sequence of any of these frameworks or any artificial framework. Preferably known frameworks are used to determine the identities of each X. In certain embodiments, the framework for determining X is that from Sp1, Sp1C or Zif268. In a preferred embodiment, the framework has the sequence of Sp1C domain 2 (Le., the middle finger of Sp1C), which sequence is -Pro-Tyr-Lys-Cys-Pro-Glu-Cys-Gly-Lys-Ser-Phe-Ser-Z⁻¹-Ser- Z²-Z³-Leu-Gln-Z⁶-His-Gln-Arg-Thr-His-Thr-Gly-Glu-Lys- (SEQ ID NO: 3). Such AZPs are more fully described in U.S. Ser. No. 09/911,261 to Takashi Sera, filed Jul. 23, 2001.

The AZPs of the invention can comprise from 3 to 40 zinc fingers, from 3 to 15 fingers, 3 to 12 fingers, 3 to 9 fingers or 3 to 6 fingers, as well as ZFPs with 3, 4, 5, 6, 7, 8 or 9 fingers.

The four nucleic acid-contacting residues of the zinc finger, designated as Z⁻¹, Z², Z³ and Z⁶ in the above formula, are primarily responsible for determining specificity and affinity of DNA binding. These four amino acid residues may also be referred to as the base-contacting amino acids. These four residues occur in the same position relative to the first consensus histidine and second consensus cysteine. The first residue is seven residues to the N-terminal side of the first consensus histidine and six residues to the C-terminal side of the second consensus cysteine. The first residue is also referred to as the “−1 position” and is so designated because it represents the residue immediately adjacent to the N-terminus of the α-helix in the zinc finger (with position 1 thus being the first N-terminal residue of the α-helix). The other three amino acids occur at positions two, three and six of the α-helix, and are referred to as the “2 position”, “3 position” and “6 position”, respectively. These four positions are interchangeably referred to herein as the Z⁻¹, Z², Z³ and Z⁶ positions.

The recognition code table provides a method to determine the identify of Z⁻¹, Z², Z³ and Z⁶ for a given nucleotide sequence. In the recognition code table (and for each 4 base-pair portion of a nucleotide sequence), the bases are always provided in 5′ to 3′ order. The fourth base, however, is always the complement of the fourth base provided in the target sequence. For example, if the target sequence is written as ATCC, then it means a sense strand target sequence of 5′-ATCC-3′ and an antisense strand of 3′-TAGG-5′. Thus, when the sense strand sequence ATCC is translated to amino acids from Table 1 below, the first base of A means there is glutarnine at position 6, the second base of T means there is serine at position 3 and the third base of C means there is glutamic acid at position −1. However, with the fourth base written as C, it means that the complement of C, i.e., G, is found in the table and used to identify the amino acid of position 2. In this case, the amino acid at position two is serine.

Tables 1 and 2 provide the preferred and alternative recognition code tables for the AZPs that are useful in the invention, respectively, in summary format:

TABLE 1

| | 1^{st} base | 2^{nd} base | 3^{rd} base | 4^{th} base |
|---|---|---|---|---|
| G | Arg | His | Arg | Ser |
| A | Gln | Asn | Gln | Asn |
| T | Thr, Tyr, Leu | Ser | Thr, Met | Thr |
| C | Glu | Asp | Glu | Asp |
| | Position 6 | Position 3 | Position −1 | Position 2 |

TABLE 2

| | 1^{st} base | 2^{nd} base | 3^{rd} base | 4^{th} base |
|---|---|---|---|---|
| G | Arg, Lys | His, Lys | Arg, Lys | Ser, Arg |
| A | Gln, Asn | Asn, Gln | Gln, Asn | Asn, Gln |
| T | Thr, Tyr, Leu, | Ser, Ala, Val, | Thr, Met, Leu, | Thr, Val, Ala |
| | Ile, Met | Thr | Ile | |
| C | Glu, Asp | Asp, Glu | Glu, Asp | Asp, Glu |
| | Position 6 | Position 3 | Position −1 | Position 2 |

In Table 2, the order of amino acids listed in each box represents, from left to right, the most preferred to least preferred amino acid at that position.

These recognition code tables can also be described as follows below. The preferred recognition code table for the AZPs (equivalent to Table 1) is, for each four base target sequence, in 5′ to 3′ order:
(i) if the first base is G, then Z is arginine,
   if the first base is A, then Z⁶ is glutamine,
   if the first base is T, then Z⁶ is threonine, tyrosine or leucine,
   if the first base is C, then Z⁶ is glutamic acid,
(ii) if the second base is G, then Z³ is histidine,
   if the second base is A, then Z³ is asparagine,
   if the second base is T, then Z³ is serine,
   if the second base is C, then Z³ is aspartic acid,
(iii) if the third base is G, then Z⁻¹ is arginine,
   if the third base is A, then Z⁻¹ is glutamine,
   if the third base is T, then Z⁻¹ is threonine or methionine,
   if the third base is C, then Z⁻¹ is glutamic acid,
(iv) if the complement of the fourth base is G, then Z² is serine,
   if the complement of the fourth base is A, then Z² is asparagine,
   if the complement of the fourth base is T, then Z² is threonine, and
   if the complement of the fourth base is C, then Z² is aspartic acid.
      In a preferred embodiment of the above recognition code (ie., the Table 1 recognition code), if the first base is T, then Z⁶ is threonine; and if the third base is T, then Z⁻¹ is threonine (Table 1).

The alternative recognition code table (equivalent to Table 2) can also be presented as follows:
(i) if the first base is G, then Z⁶ is arginine or lysine,
   if the first base is A, then Z⁶ is glutamine or asparagine,
   if the first base is T, then Z⁶ is threonine, tyrosine, leucine, isoleucine or methionine,
   if the first base is C, then Z⁶ is glutamic acid or aspartic acid,
(ii) if the second base is G, then Z³ is histidine or lysine,
   if the second base is A, then Z³ is asparagine or glutamine,
   if the second base is T, then Z³ is serine, alanine, valine or threonine
   if the second base is C, then Z³ is aspartic acid or glutamic acid,
(iii) if the third base is G, then Z⁻¹ is arginine or lysine,
   if the third base is A, then Z⁻¹ is glutamine or asparagine,
   if the third base is T, then Z^{—1} is threonine, methionine leucine or isoleucine,
   if the third base is C, then Z⁻¹ is glutamic acid or aspartic acid,
(iv) if the complement of the fourth base is G, then Z² is serine or arginine,
   if the complement of the fourth base is A, then Z² is asparagine or glutamine,
   if the complement of the fourth base is T, then Z² is threonine, valine or alanine, and
   if the complement of the fourth base is C, then Z² is aspartic acid or glutamic acid.

To use the recognition code table to design and identify an AZP for a given nucleotide sequence, a nucleotide sequence of length 3N+1 base pairs, wherein N is the number of overlapping 4 base pair segments in the target, is divided into overlapping 4 base pair segments, where the fourth base of each segment, up to the N−1 segment, is the first base of the immediately following segment. The identities of each Z⁻¹, Z², Z³ and Z6 in the zinc finger are then determined according to the recognition code table.

Zinc fingers designed in accordance with this invention are either covalently joined directly to one another or can be separated by a linker of from 1-10 amino acids. The linker amino acids can provide flexibility or some degree of structural rigidity. The choice of linker can be, but is not necessarily, dictated by the desired affinity of the ZFP for its cognate nucleotide sequence. It is within the skill of the art to test and optimize various linker sequences to improve the binding affinity of the AZP for its cognate target sequence. For example, one useful arrangement for a six finger ZFP, is to have the first three zinc fingers be joined without amino acid linkers, a flexible amino acid linker between the third and fourth fingers and the last three fingers joined without amino acid linkers. This arrangement appears to allow each three finger group to independently bind its target sequence while minimizing steric hindrance for the binding of the other three finger group.

In an embodiment, longer genomic sequences are targeted using multi-finger AZPs linked to other multi-fingered AZPs using flexible linkers including, but not limited to, GGGGS, GGGS and GGS (these sequences can be part of the 1-10 additional amino acids in the AZPs; SEQ ID NO: 4, residues 2-5 of SEQ ID NO: 4; and residues 3-5 of SEQ ID NO: 4, respectively).

In addition, the nucleic acid binding domain of the chimeric proteins of the invention can be designed to bind to non-contiguous nucleotide sequences, either using a single domain or multiple domains. For example, the nucleotide sequence bound by a six-finger AZP can be a ten base pair sequence (recognized by three fingers) with intervening bases (that do not contact the zinc fingers) and a second ten base pair sequence (recognized by the other three fingers). The number of intervening bases can vary, such that one can compensate for this intervening distance with an appropriately designed amino acid linker between the two three-finger parts of the AZP. A range of intervening nucleic acid bases in a target binding site can be from 5-100, and preferably from 10-20 or less bases, more preferably 10 or less, and even more preferably 6 or less bases. Of course, the linker maintains the reading frame between the linked parts of the AZP.

Methods of designing and constructing nucleic acids encoding ZFPs and AZPs by phage display, random mutagenesis, combinatorial libraries, computer/rational design, affinity selection, PCR, cloning from cDNA or genomic libraries, synthetic construction and the like are known. (see, e.g., U.S. Pat. No. 5,786,538; Wu et al., Proc. Natl. Acad. Sci. USA 92:344-348 (1995); Jamieson et al., Biochemistry 33:5689-5695 (1994); Rebar & Pabo, Science 263:671-673 (1994); Choo & Klug, Proc. Natl. Acad. Sci. USA 91: 11168-11172 (1994); Desjarlais et al., Proc. Natl. Acad. Sci. USA 89:7345-5349 (1992); Desjarlais et al., Proc. Natl. Acad. Sci. USA 90:2256-2260 (1993); Desjarlais et al., Proc. Natl. Acad. Sci. USA 91:11099-11103; Pomerantz et al., Science 267:93-96 (1995); Pomerantz et al., Proc. Natl. Acad. Sci. USA 92:9752-9756 (1995); Liu et al., Proc. Natl. Acad. Sci. USA 94:5525-5530 (1997); Griesman & Berg, Science 275:657-661 (1997); and U.S. Ser. No. 09/911,261 to Sera filed Jul. 23, 2001 (the Sera application). For example, the Sera application describes a modular method of making AZPs that can be adapted to produce combinatorial libraries of AZPs. These AZPs can then be used in screening and/or selection assays to identify AZPs that bind at or near a target gene. Once such AZPs are known, they can serve as the first domain of the chimeric proteins of the invention. Similarly, any AZP (or ZFP) obtained by a screening or selection procedure, whether an in vitro or in vivo procedure, can be used as the first domain, provided that the AZP (or ZFP) specifically binds to or associates with a target gene in the manner contemplated by the invention.

In accordance with the invention, the chimeric proteins of the invention can have multiple first, nucleic acid binding domains. Each such domain specifically binds to a selected nucleotide sequence. Such sequences can be near one another or located at some distance provided that the distance does not prevent the chimeric protein from being localized to the nuclear periphery and repressing expression of the associated target gene or genes. When one first domain is present, the nucleotide sequence can be at any location relative to the intended target gene, provided that binding or association of the chimeric protein with both the nucleotide sequence and the nuclear periphery represses gene expression. Additional first domains can be added to the chimeric proteins of the invention to enhance transcriptional repression. The chimeric proteins have from one to six first domains, from one to three first domains, or one first domain.

Examples of other transcriptional repressors include, but are not limited to, the KRAB repression domain from the human KOX-I protein (Thiesen et al., New Biologist 2:363-374 (1990); Margolin et al., Proc. Natl. Acad. Sci. U.S.A. 91:4509-4513 (1994); Pengue et al., Nucl. Acids Res. 22:2908-2914 (1994); Witzgall et al., Proc. Natl. Acad. Sci. U.S.A. 91:4514-4518 (1994)). KAP-1, a KRAB co-repressor, can be used with KRAB (Friedman et al., Genes Dev. 10:2067-2078 (1996)). KAP-I can also be used alone. Other transcription factors and transcription factor domains that act as transcriptional repressors include MAD (see, e.g., Sommer et al., J. Biol. Chem. 273:6632-6642 (1998); Gupta et al., Oncogene 16:1149-1159 (1998); Queva et al., Oncogene 16:967-977 (1998); Larsson et al., Oncogene: 737-748 (1997); Laherty et al., Cell 89:349-356 (1997); and Cultraro et al., Mol. Cell. Biol. 17:2353-2359 (19977)); FKHR (forkhead in rhapdosarcoma gene; Ginsberg et al., Cancer Res. 15:3542-3546 (1998); Epstein et al., Mol. Cell. Biol. 18:4118-4130 (1998)); EGR-I (early growth response gene product-1; Yan et al., Proc. Natl. Acad. Sci. U.S.A. 95:8298-8303 (1998); and Liu et al., Cancer Gene Ther. 5:3-28 (1998)); the ets2 repressor factor repressor domain (ERD; Sgouras et al., EMBO J. 14:4781-4793 ((19095)); and the MAD smSIN3 interaction domain (SID; Ayer et al., Mol. Cell. Biol. 16:5772-5781 (1996)).

The second domain of the chimeric proteins of the invention is capable of associating with the nuclear periphery. This association may be direct or indirect and is typically mediated by protein-protein interactions between the second domain and one or more protein components of the nuclear envelope, the nuclear lamina, heterochromatin or any combination thereof. For example, the second domain(s) can associate with or bind to (1) a protein that is a component of the nuclear lamina or (2) a another protein that associates with a protein that is a component of the nuclear lamina. Hence, the second domain can comprise a nuclear envelope-binding protein, nuclear lamina-binding protein (alternatively known as a lamina-associated polypeptide), a heterochromatin-binding protein, the binding moiety of any of these proteins, a protein capable of associating with or binding to any of the foregoing, or any combination thereof.

The nuclear envelope- and nuclear lamina-binding proteins (or the appropriate binding moiety thereof) are known to or engineered to interact, respectively, with the nuclear envelope (particularly the inner nuclear membrane) or the nuclear lamina by binding directly or indirectly to those structural components of the nucleus. In some cases, the second domain of the chimeric protein may interact with both the inner nuclear membrane and the nuclear lamina. Preferred nuclear envelope- and/or nuclear lamina-binding proteins (or their binding moiety) include, but are not limited to, lamins (e.g., lamins A, B and C) and lamina-binding proteins such as LAP 2β and the LAP 2β interaction region (amino acids 138-524). (Nili et al., 2001]. Other preferred proteins for the second domain include the 524-amino acid mouse GCL protein [Leatherman et al. (2000) Mech. Dev. 92: 145-153], or any other GCL protein such as from Drosophila or any other mammalian species. GCL appears to bind indirectly to the nuclear lamina via a lamina-associated protein (LAP). Other useful proteins (or their binding moieties) for the second domain include the hyperphosphorylated form of Rb, Oct-1 and the insulin activator IPF/PDX-1 (which in the presence of low glucose is localized to the nuclear membrane). For all second domains, it may be useful to select a domain that is from the same species as the target gene. Heterochromatin-binding proteins (or the moieties thereof with binding activity) can also be used as second domains in the chimeric proteins of the invention. Useful heterochromatin-binding proteins include, but are not limited to, HP1 and polycomb-group proteins.

In another aspect of the invention, a nuclear localization peptide can be attached to the chimeric proteins of the invention to aid in transporting that protein to the nuclear compartment. The nuclear localization peptide facilitates the transport of proteins present in the cytoplasm into the nucleus. The nuclear localization peptide can be used alone or on conjunction with other domains. One example of a nuclear localization peptide is a peptide from the SV40 large T antigen having the sequence Pro-Lys-Lys-Lys-Arg-Lys-Val (SEQ ID NO: 9).

In addition, the chimeric proteins can have a cellular uptake signal attached, either alone or in conjunction a nuclear localization peptide, to aid in transport of the protein into the cell. Such cellular uptake signals include, but are not limited to,
the minimal Tat protein transduction domain which is residues 47-57 of the human immunodeficiency virus Tat protein: YGRKKRRQRRR (SEQ ID NO: 5);
residues 43-58 of the Antenapedia (pAntp) homeodomain: Arg-Gln-Ile-Lys-Ile-Trp-Phe-Gln-Asn-Arg-Arg-Met-Lys-Trp-Lys-Lys (SEQ ID NO: 10) (Derossi et al., (1994) J. Biol. Chem. 269:10444-10450);
residues 267-300 of the herpes simplex virus (HSV) VP22 protein: Asp-Ala-Ala-Thr-Ala-Thr-Arg-Gly-Arg-Ser-Ala-Ala-Ser-Arg-Pro-Thr-Glu-Arg-Pro-Arg-Ala-Pro-Ala-Arg-Ser-Ala-Ser-Arg-Pro-Arg-Arg-Pro-Val-Glu (SEQ ID NO: 11) (Elliott et al. (1997) Cell 88:223-233);
various basic peptides with reported cellular uptake signal activity such as Tyr-Ala-Arg-Ala-Ala-Ala-Arg-Gln-Ala-Arg-Ala (SEQ ID NO: 12)(Ho et al. (2001) Cancer Res. 61:474-477), Arg-Arg-Arg-Arg-Arg-Arg-Arg-Arg-Arg (SEQ ID NO: 13), also known as R9 (Jin et al. (2001) Free Rad. Biol. Med. 31:1509-1519) and the all D-arginine form of R9 (Winder et al. (2000) Proc. Natl. Acad. Sci. USA 97:13003-13008); and
the peptides described by the Temsamani group which include the peptides capable of carrying substances across the blood brain barrier of WO00/32236, the peptides capable of carrying an anti-cancer agent into a cancer cell as described in WO00/32237, the amphipathic peptide moieties of the antibiotic peptides of WO02/02595, the amphipathic peptides for transporting negatively charged substances into cells or cell nuclei as described in WO02/053583, and the peptide vector moieties of the analgesic molecules of WO02/067994. The peptides described by Temsamnani, include but are not limited, to D-penetratin (rqikiwfqnrrmkwkk; all amino acids being in the D form) (SEQ ID NO: 14), pAntp and active variants thereof, SynB1 (RGGRLSYSRRRFSTSTGR) (SEQ ID NO: 15), L-SynB3 (RRLSYSRRRF) (SEQ ID NO: 16), and D-SynB3 (rrlsysrrrf; all amino acids being in the D form) (SEQ ID NO: 17).

For ease of purification, monitoring expression, or monitoring cellular and subcellular localization, a chimeric protein of the invention can also be expressed as a fusion protein with such proteins or protein moieties as maltose binding protein (“MBP”), green fluorescent protein (GFP), glutathione S transferase (GST), hexahistidine, c-myc, and the FLAG epitope Asp-Tyr-Lys-Asp-Asp-Asp-Asp-Lys (SEQ ID NO: 18).

The chimeric proteins of the invention can be prepared either synthetically or recombinantly, preferably recombinantly, using any of the multitude of techniques well-known in the art. When the proteins are prepared recombinantly, e.g., via a DNA encoding the chimeric protein, the codon usage can be optimized for high expression in the organism in which that protein is to be expressed. Such organisms include bacteria, fungi, yeast, animals, insects and plants. More specifically the organisms, include but are not limited to, human, mouse, E. coli, cereal plants, rice, tomato and corn. When the nucleic acid will be used to deliver the chimeric protein of the invention, codon usage can also be optimized for the eukaryotic organism which will receive the nucleic acid construct.

Any suitable method of protein purification known to those of skill in the art can be used to purify the chimeric proteins of the invention [see, e.g., Sambrook et al. (1989) Molecular Cloning: A Laboratory Manual (2d ed., Cold Spring Harbor Laboratory Press, Plainview, N.Y.]. In addition, any suitable host can be used for protein expression, e.g., bacterial cells, insect cells, yeast cells, mammalian cells, plant cells and the like.

The chimeric proteins of the invention and the nucleic acids encoding same are used to repress, down regulate or decrease gene expression of a target gene (as determined by its association with a particular nucleotide sequence) in any eukaryotic organism, including yeast, animals and plants. The target gene can encode any eukaryotic gene for which repression of expression is desired. For example, target genes can encode cytokines, interleukins, oncogenes, angiogenesis factors, anti-angiogenesis factors, drug resistance proteins, growth factors and/or tumor suppressors. The target gene can also be a viral gene, particularly from DNA viruses. The target gene can encode a plant gene. Preferred sources of those plant genes are from tomato, corn, rice and cereal plants.

The target genes can be oncogenes, including, but not limited to, myc, jun, fos, myb, max, mad, rel, ets, bcl, myb, mos family members and their associated factors and modifiers. Oncogenes are described in, for example, Cooper, Oncogenes, 2nd ed., The Jones and Bartlett Series in Biology, Boston, Mass., Jones and Bartlett Publishers, 1995. The ets transcription factors are reviewed in Waslylk et al., Eur. J. Biochem. 211:7-18 (1993). Myc oncogenes are reviewed in, for example, Ryan et al., Biochem. J. 314:713-21 (1996). The Jun and fos transcription factors are described in, for example, The Fos and Jun Families of Transcription Factors, Angel & Herrlich, eds. (1994). The max oncogene is reviewed in Hurlin et al., Cold Spring Harb. Symp. Quant. Biol. 59:109-16. The myb gene family is reviewed in Kanei-Ishii et al., Curr. Top. Microbiol. Immunol. 211:89-98 (1996). The mos family is reviewed in Yew et al., Curr. Opin. Genet. Dev. 3:19-25 (1993).

The chimeric proteins of the present invention can be used to inhibit the expression of a disease-associated genes. In one example, the disease-associated gene is an oncogene such as a BCR-ABL fusion oncogene or a ras oncogene, and the DNA binding domain is designed to bind to the DNA sequence GCAGAAGCC (SEQ ID NO: 6) and is capable of inhibiting the expression of the BCR-ABL fusion oncogene by both targeting to the nuclear periphery and by inhibiting expression by binding to a sequence needed in the transcription process. Transcription factors involved in disease are reviewed in Aso et al., J Clin. Invest. 97:1561-9 (1996).

B. Methods of Use

Another aspect of the invention relates to a method of repressing or down regulating expression of a target gene by localizing the gene to the nuclear periphery. This method involves contacting the target nucleic acid containing a nucleotide sequence associated with or in sufficient proximity to the target gene with a chimeric protein of the invention. The nucleic acid can be present in a cell or in an organism and is preferably genomic DNA. However, the nucleic acid can also be extrachromosomal DNA present in the nucleus. The nucleotide sequence and target gene are as described hereinabove. The proximity of the nucleotide sequence to the target gene is sufficient to allow measurable repression or down regulation of the target gene after exposure to a chimeric protein of the invention.

In accordance with the invention, the chimeric protein can be introduced into a cell as a protein or as a nucleic acid encoding that protein. When protein is used, the chimeric protein can, optionally, have a cellular-uptake signal and/or a nuclear localization signal to facilitate uptake of the protein by the cell and its transport into the nucleus. The amount of the chimeric protein needed to repress or down regulate expression of the target gene can be readily determined by those of skill in the art. When a nucleic acid such as RNA or DNA is used, it can be delivered in any of a variety of forms, including as naked plasmid or other DNA, formulated in liposomes, in a viral vector (including RNA viruses and DNA viruses), via a pressure injection apparatus such as the Powdedject™ system using RNA or DNA, or by any other convenient means. Again, the amount of nucleic acid needed to repress or down regulate expression of the target gene can be readily determined by those of skill in the art based on the target cell or organism, the delivery formulation and mode and whether the nucleic acid is DNA or RNA. Preferably DNA is used.

In accordance with the invention, the chimeric protein binds to the target nucleic acid with a chimeric protein at the nucleotide sequence associated with a target gene. Assays to determine whether binding has occurred and the efficiency by which the repression of the target gene or protein of interest occurs are known. In brief, in one embodiment, a reporter gene such as ∃-glucuronidase (GUS), chloramphenicol acetyl transferase (CAT), β-galactosidase (β-gal) or green fluorescent protein (GFP) is operably linked to the target gene sequence controlling promoter, ligated into a transformation vector, and transformed into an animal or plant cell. After introduction of the chimeric protein (whether as a protein or as a nucleic acid which is translated to produce the protein) the level of the reporter gene can be assessed relative to the appropriate controls. As an alternative, levels of RNA can be measured by a Northern blot or other means. This latter method is useful when reporter constructs are not practical.

The invention contemplates gene regulation which may be tissue specific or not, inducible or not, and which may occur in animal cells, yeast cells, insect cells, or plant cells either in culture or in intact plants. Useful repression levels can vary, depending on how tightly the target gene is normally regulated, the effects of changes in regulation, and other similar factors. Desirably, the change in gene expression is modified by at least about 1.5-fold to 2-fold; about 3-fold to 5-fold; about 8- to 10- to 15-fold; or even more such as 20- to 25- to 30-fold; and even 40-, 50-, 75-, or 100-fold, or more. The degree of change in gene expression again varies from system to system

“Organisms” as used are any eukaryotic organism including yeast, animals, birds, insects, plants and the like. Animals include, but are not limited to, mammals (humans, primates, etc.), commercial or farm animals (fish, chickens, cows, cattle, pigs, sheep, goats, turkeys, etc.), research animals (mice, rats, rabbits, etc.) and pets (dogs, cats, parakeets and other pet birds, fish, etc.). As contemplated herein, particular animals may be members of multiple animal groups.

The chimeric proteins of the present invention (or nucleic acids encoding those proteins) can be used, for example, to repress, down regulate or decrease gene expression, over a broad range of plant types and plant tissue, preferably the class of higher plants amenable to transformation techniques, particularly monocots and dicots.

A “plant” refers to any plant or part of a plant at any stage of development, including seeds, suspension cultures, embryos, meristematic regions, callus tissue, leaves, roots, shoots, gametophytes, sporophytes, pollen, and microspores, and progeny thereof. Also included are cuttings, and cell or tissue cultures. As used in conjunction with the present invention, the term “plant tissue” includes, but is not limited to, plant cells, plant organs (e.g., leafs, stems, roots, meristems) plant seeds, protoplasts, callus, cell cultures, and any groups of plant cells organized into structural and/or functional units.

Particularly preferred are monocots such as the species of the Family Gramineae including Sorghum bicolor and Zea mays. The isolated nucleic acid and proteins of the present invention can also be used in species from the genera: Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Avena, Hordeum, Secale, and Triticum.

Preferred plants and plant tissue includes those from corn (Zea mays), canola (Brassica napus, Brassica rapa ssp.), alfalfa (Medicago sativa), rice (Oryza sativa), rye (Secale cereale), sorghum (Sorghum bicolor, Sorghum vulgare), sunflower (Helianthus annuus), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato Qpomoea batatus), cassava (Manihot esculenta), coffee (Cqfea spp.), coconut (Cocos nucijra), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integr˜fblia), almond (Prunus amygdalus), sugar beets (Beta vulgaris), sugarcane (Saccharum spp.), duckweed (Lemna spp.), oats, barley, vegetables, ornamentals, and conifers.

Preferred vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucurnis such as cucumber (C. sativus), cantaloupe (C cantalupensis), and musk melon (C. melo).

Preferred ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbiapulcherrima), and chrysanthemum.

Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Isuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis).

Most preferably, plants and plant tissue of the present invention are crop plants (for example, corn, alfalfa, sunflower, canola, soybean, cotton, peanut, sorghum, wheat, tobacco, etc.), even more preferably corn and soybean plants, yet more preferably corn plants.

As used herein, “transgenic plant” or “genetically modified plant” includes reference to a plant which comprises within its genome a heterologous polynucleotide (i.e., a polynucleotide from a source other than the recipient organism). Generally, and preferably, the heterologous polynucleotide is stably integrated within the genome such that the polynucleotide is passed on to successive generations. The heterologous polynucleotide may be integrated into the genome alone or as part of a recombinant expression cassette. “Transgenic” is used herein to include any cell, cell line, callus, tissue, plant part or plant, the genotype of which has been altered by the presence of heterologous nucleic acid including those transgenics initially so altered as well as those created by sexual crosses or asexual propagation from the initial transgenic. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

C. Expression Systems

The present invention also provides recombinant expression cassettes comprising a chimeric protein-encoding nucleic acid of the present invention. A nucleic acid sequence coding for the desired polynucleotide of the present invention can be used to construct a recombinant expression cassette which can be introduced into a desired host cell. A recombinant expression cassette will typically comprise a polynucleotide of the present invention operably linked to transcriptional initiation regulatory sequences which will direct the transcription of the polynucleotide in the intended host cell, such as tissues of a transformed plant. The expression vectors can be a mammalian expression vector, an insect expression vector, a yeast expression vector or a plant expression vector. When the protein is being expressed for the purpose of preparing and purifying the protein (which can then be used, e.g., in the methods of the invention), the expression vector can be a bacterial expression vectors. Expression vectors are well known in the art and can be readily selected for the desired purpose.

The elements for transcription include but are not limited to promoters active in eukaryotic cells, enhancers, transcription termination signals including polyadenylation signals or polyA tracts, elements to facilitate nucleocytoplasmic transport, and the like. Suitable transcription termination elements include the SV 40 transcription termination region and terminators derived therefrom.

Any mammalian, yeast, bacterial, insect, viral, other eukaryotic expression vector or expression cassette can be employed in the present invention and can be selected from, e.g., any of the many commercially available vectors or cassettes, such as pCEP4 or pRc/RSV obtained from Invitrogen Corporation (San Diego, Calif.), pXT1, pSG5, pPbac or pMbac obtained from Stratagene (La Jolla, Calif.), pPUR or pMAM obtained from ClonTech (Palo Alto, Calif.), and pSVβ-gal obtained from Promega Corporation (Madison, Wis.), or synthesized either de novo or by adaptation of a publically or commercially available eukaryotic expression system.

The individual elements within the expression cassette can be derived from multiple sources and may be selected to confer specificity in sites of action or longevity of the cassettes in the recipient cell. Such manipulation of the expression cassette can be done by any standard molecular biology approach.

Plant expression vectors may include (1) a cloned plant gene under the transcriptional control of 5′ and 3′ regulatory sequences and (2) a dominant selectable marker. Such plant expression vectors may also contain, if desired, a promoter regulatory region (e.g., one conferring inducible or constitutive, environmentally- or developmentally-regulated, or cell- or tissue-specific/selective expression), a transcription initiation start site, a ribosome binding site, an RNA processing signal, a transcription termination site, and/or a polyadenylation signal.

Typical vectors useful for expression of genes in higher plants are well known in the art and include vectors derived from the tumor-inducing (Ti) plasmid of Agrobacterium tumefaciens described by Rogers et al., Meth. in Enzymol., 153:253-277 (1987). These vectors are plant integrating vectors in that on transformation, the vectors integrate a portion of vector DNA into the genome of the host plant. Exemplary A. tumefaciens vectors useful herein are plasmids pKYLX6 and pKYLX7 of Schardl et al., Gene, 6 1: 1-11 (1987) and Berger et al., Proc. Natl. Acad. Sci. U.S.A., 86:8402-8406 (1989). Another useful vector is plasmid pBI101.2.

Cell transformation techniques and gene delivery methods (such as those for in vivo use to deliver genes) are well known in the art. Any such technique can be used to deliver a nucleic acid encoding the chimeric proteins of the invention to a cell or in vivo to the cells of a subject, respectively.

The term “expression cassette” as used herein means a DNA sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least one of its components is heterologous with respect to at least one of its other components. The expression cassette may also be one which is naturally occurring but has been obtained in a recombinant form useful for heterologous expression. Typically, however, the expression cassette is heterologous with respect to the host, ie., the particular DNA sequence of the expression cassette does not occur naturally in the host cell and must have been introduced into the host cell or an ancestor of the host cell by a transformation event. The expression of the nucleotide sequence in the expression cassette may be under the control of a constitutive promoter or of an inducible promoter which initiates transcription only when the host cell is exposed to some particular external stimulus. In the case of a multicellular organism, such as a plant, the promoter can also be specific to a particular tissue or organ or stage of development.

Various promoters well-known to be useful for driving expression of genes in animal cells, such as the viral-derived SV40, CMV immediate early and, RSV promoters or eukaryotic derived ∃-casein, uteroglobin, ∃-actin or tyrosinase promoters. The particular promoter is not critical to the invention, unless the object is to obtain temporal- or tissue-specific expression. For example, a promoter can be selected which is only active in the desired tissue or selected cell type. Examples of tissue-specific promoters include, but are not limited to, ∀ S1- and 3-casein promoters which are specific for mammary tissue (Platenburg et al., Trans. Res., 3:99-108 (1994); and Maga et al., Trans. Res., 3:36-42 (1994)); the phosphoenolpyruvate carboxykinase promoter which is active in liver, kidney, adipose, jejunum and mammary tissue (McGrane et al., J. Reprod. Fert., 41:17-23 (1990)); the tyrosinase promoter which is active in lung and spleen cells, but not testes, brain, heart, liver or kidney (Vile et al., Canc. Res., 54:6228-6234 (1994)); the involucerin promoter which is only active in differentiating keratinocytes of the squamous epithelia (Carroll et al., J. Cell Sci., 103:925-930 (1992)); and the uteroglobin promoter which is active in lung and endometrium (Helftenbein et al., Annal. N.Y. Acad. Sci., 622:69-79 (1991)).

Alternatively, cell specific enhancer sequences can be used to control expression, for example human neurotropic papovirus JCV enhancer regulates viral transcription in glial cells alone (Remenick et al., J. Virol., 65:5641-5646 (1991)). Yet another way to control tissue specific expression is to use a hormone responsive element (HRE) to specify which cell lineages a promoter will be active in, for example, the MMTV promoter requires the binding of a hormone receptor, such as progesterone receptor, to an upstream HRE before it is activated (Beato, FASEB J., 5:2044-2051 (1991); and Truss et al., J. Steroid Biochem. Mol. Biol., 41:241-248 (1992)).

A plant promoter fragment can be employed which will direct expression of a polynucleotide of the present invention in all tissues of a regenerated plant. Such promoters are referred to herein as “constitutive” promoters and are active under most environmental conditions and states of development or cell differentiation. Examples of constitutive promoters include the cauliflower mosaic virus (CaMV) 35S transcription initiation region, the P- or 2′ - promoter derived from T-DNA of Agrobacterium tumefaciens, the ubiquitin I promoter, the Smas promoter, the cinnamyl alcohol dehydrogenase promoter (U.S. Pat. No. 5,683,439), the Nos promoter, the pEmu promoter, the nibisco promoter, the GRP 1-8 promoter, and other transcription initiation regions from various plant genes known to those of skill in the art.

Alternatively, the plant promoter can direct expression of a polynucleotide of the present invention in a specific tissue or may be otherwise under more precise environmental or developmental control. Such promoters are referred to here as “inducible” promoters. Environmental conditions that may effect transcription by inducible promoters include pathogen attack, anaerobic conditions, or the presence of light. Examples of inducible promoters include the AdhI promoter which is inducible by hypoxia or cold stress, the Hsp70 promoter which is inducible by heat stress, and the PPDK promoter which is inducible by light. Examples of promoters under developmental control include promoters that initiate transcription only, or preferentially, in certain tissues, such as leaves, roots, fruit, seeds, or flowers. An exemplary promoter is the anther specific promoter 5126 (U.S. Pat. Nos. 5,689,049 and 5,689,051). The operation of a promoter may also vary depending on its location in the genome. Thus, an inducible promoter may become fully or partially constitutive in certain locations.

Both heterologous and non-heterologous (i.e., endogenous) promoters can be employed to direct expression of the nucleic acids of the present invention. These promoters can also be used, for example, in recombinant expression cassettes to drive expression of antisense nucleic acids to reduce, increase, or alter concentration and/or composition of the proteins of the present invention in a desired tissue. Thus, in some embodiments, the nucleic acid construct will comprise a promoter functional in a plant cell, such as in Zea mays, operably linked to a polynucleotide of the present invention. Promoters useful in these embodiments include the endogenous promoters driving expression of a polypeptide of the present invention.

In some embodiments, isolated nucleic acids which serve as promoter or enhancer elements can be introduced in the appropriate position (generally upstream) of a non-heterologous form of a polynucleotide so as to up or down regulate its expression. For example, endogenous promoters can be altered in vivo by mutation, deletion, and/or substitution (U.S. Pat. No. 5,565,350; PCT/US93/03868), or isolated promoters can be introduced into a plant cell in the proper orientation and distance from a gene of the present invention so as to control the expression of the gene. Gene expression can be modulated under conditions suitable for plant growth so as to alter the total concentration and/or alter the composition of the polypeptides of the present invention in plant cell.

A variety of promoters will be useful in the invention, particularly to control the expression of the chimeric proteins, the choice of which will depend in part upon the desired level of protein expression and desired tissue-specific, temporal specific, or environmental cue-specific control, if any in a plant cell. Constitutive and tissue specific promoters are of particular interest. Such constitutive promoters include, for example, the core promoter of the Rsyn7, the core CAMV 35S promoter (Odell et al. (1985) Nature 313:810-812), rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689), pEMU (Last et al. (1991) Theor. Appl. Genet. 81:581-588), MAS (Veltenet al. (1984) EMBO J. 3:2723-2730), and constitutive promoters described in, for example, U.S. Pat. Nos. 5,608,149; 5,608,144; 5,604,121; 5,569,597; 5,466,785; 5,399,680; 5,268,463; and 5,608,142.

Tissue-specific promoters can be utilized to target enhanced expression within a particular plant tissue. Tissue-specific promoters include those described by Yamamoto et al. (1997) Plant J. 12(2)255-265, Kawamata et al. (1997) Plant Cell Physiol. 38(7):792-803, Hansen et al. (1997) Mol. Gen Genet. 254(3):337), Russell et al. (1997) Transgenic Res. 6(2):15 7-168, Rinehart et al. (1996) Plant Physiol. 112(3):1331, Van Camp et al. (1996) Plant Physiol. 112(2):525-535, Canevascini et al. (1996) Plant Physiol. 112(2):513-524, Yamamoto et al. (1994) Plant Cell Physiol. 35(5):773-778, Lam (1994) Results Probl. Cell Differ. 20:181-196, Orozco et al. (1993) Plant Mol. Biol. 23 (6):1129-113 8, Matsuoka et al. (1993) Proc Natl. Acad. Sci. USA 90(20):9586-9590, and Guevara-Garcia et al. (1993) Plant J. 4(3):495-505. Such promoters can be modified, if necessary, for weak expression.

Leaf-specific promoters are known in the art, and include those described in, for example, Yamamoto et al. (1997) Plant J. 12(2):255-265, Kwon et al. (1994) Plant Physiol. 105:357-67, Yamamoto et al. (1994) Plant Cell Physiol. 35(5):773-778, Gotor et al. (1993) Plant J. 3:509-18, Orozco et al. (1993) Plant Mol. Biol. 23(6):1129-1138, and Matsuoka et al. (1993) Proc. Natl. Acad. Sci. U.S.A .90(20):9586-9590.

Any combination of constitutive or inducible and non-tissue specific or tissue specific may be used to control the expression of the chimeric proteins of the invention. The desired control may be temporal, developmental or environmentally controlled using the appropriate promoter. Environmentally controlled promoters are those that respond to assault by pathogen, pathogen toxin, or other external compound (e.g., intentionally applied small molecule inducer). An example of a temporal or developmental promoter is a fruit ripening-dependent promoter. Particularly preferred are the inducible PRI promoter, the maize ubiquin promoter, and ORS.

Methods for identifying promoters with a particular expression pattern, in terms of, e.g., tissue type, cell type, stage of development, and/or environmental conditions, are well known in the art. See, e.g., The Maize Handbook, Chapters 114-115, Freeling and Walbot, Eds., Springer, New York (1994); Corn and Corn Improvement, Pedition, Chapter 6, Sprague and Dudley, Eds., American Society of Agronomy, Madison, Wis. (1988).

Plant transformation protocols as well as protocols for introducing nucleotide sequences into plants may vary depending on the type of plant or plant cell, i.e., monocot or dicot, targeted for transformation. Suitable methods of introducing nucleotide sequences into plant cells and subsequent insertion into the plant genome include microinjection (Crossway et al. (1986) Biotechniques 4:320-334), electroporation (Riggs et al. (1986) Proc. Natl. Acad Sci. USA 83:5602-5606, Agrobacterium-mediated transformation (Townsend et al., U.S. Pat. No. 5,563,055), direct gene transfer (Paszkowski et al. (1984) EMBO J. 3:2717-2722), and ballistic particle acceleration (see, for example, Sanford et al., U. S. Pat. No. 4,945,050; Tomes et al. (1995) “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); and McCabe et al. (1988) Biotechnology 6:923-926). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) BioTechnology 6:923-926 (soybean); Finer and McMullen (199 1) In Vitro Cell Dev. Biol. 2 7P: 175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad Sci. USA 85:4305-4309 (maize); Klein et al. (1988) Biotechnology 6:559-563 (maize); Tomes, U.S. Pat. No. 5,240,855; Buising et al., U.S. Pat. Nos. 5,322, 783 and 5,324,646; Tomes et al. (1995) “Direct DNA Transfer into Intact Plant Cells via Microprojectile Bombardment,” in Plant Cell, Tissue, and Organ Culture: Fundamental Methods, ed. Gamborg (Springer-Verlag, Berlin) (maize); Klein et al. (198 8) Plant Physiol. 91:440-444 (maize); Fromm et al. (1990) Biotechnology 8:833-839 (maize); Hooykaas-Van Slogteren et al. (1984) Nature (London) 311:763-764; Bowen et al., U.S. Pat. No. 5,736,369 (cereals); Bytebier et al. (1987) Proc. Natl. Acad Sci. USA 84:5345-5349 (Liliaceae); De Wet et al. (1985) in The Experimental Manipulation of Ovule Tissues, ed. Chapman et al. (Longman, N.Y.), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al; (1996) Nature Biotechnology 14:745-750 (maize via Agrobacterium tumefaciens); all of which are herein incorporated by reference.

The modified plant may be grown into plants by conventional methods. See, for example, McCormick et al. (1986) Plant Cell. Reports :81-84. These plants may then be grown, and either pollinated with the same transformed strain or different strains, and the resulting hybrid having the desired phenotypic characteristic identified. Two or more generations may be grown to ensure that the subject phenotypic characteristic is stably maintained and inherited and then seeds harvested to ensure the desired phenotype or other property has been achieved.

D. Molecular Switch Systems for Gene Repression

Another aspect of the invention relates to molecular switch systems for controlling gene expression, and particularly molecular switch systems for repressing or down regulating gene expression using the domains in the chimeric proteins of the inventions. Such systems (also called “chemical switches”) provide a further tool to manipulate the timing of or location where gene expression is being regulated or controlled. Briefly, the molecular switch system introduces two fusion proteins, one with the nucleic acid binding domain and the other with the nuclear periphery-binding domain, into a cell or organism. These two fusion proteins each have a second domain which specifically bind to one or the other moiety of a divalent ligand. Upon introduction of the divalent ligand into the cell or organism containing the two fusion proteins, the ligand acts as a switch to trigger formation of a complex among the three entities. This complex is then similar in function to the chimeric proteins of the invention since once formed it can bring a target gene into association with the nuclear periphery to repress or down regulate gene expression.

An example is a complex formed by a divalent chemical ligand having moieties A and B, a first fusion protein encoding an AZP and an antibody specific for moiety A (or an active fragment of such antibody) and a second fusion protein encoding the domain capable of associating with the nuclear periphery and an antibody specific for moiety B (or an active fragment of such antibody). The two fusion proteins can be separately or coordinately expressed in the same cell. Upon addition to the cell or organism of the divalent chemical that includes moiety A and moiety B linked together, the affinity of each fusion protein for either moiety A or moiety B mediates formation of a complex.

Accordingly, the first fusion protein of this aspect of the invention comprises a first domain capable of specifically binding a nucleotide sequence associated with a target gene, and a second domain capable of specifically binding to a first binding moiety of a divalent ligand, said ligand capable of uptake by a cell, wherein the first and second domains are heterologous with respect each other. The first domain of the fusion protein is the same as the first domain of the chimeric proteins of the invention. For example, this first domain of the first fusion protein can be a ZFP, an AZP, a leucine zipper protein, a helix-turn-helix protein, a helix-loop-helix protein, a homeobox domain protein, the DNA binding moiety of any of those proteins, or any combination thereof.

Likewise, the nucleotide sequence associated with the target gene, and the target gene is the same as described for the chimeric proteins of the invention.

The second fusion protein of this aspect of the invention comprises a first domain capable of associating with the nuclear periphery and a second domain capable of specifically binding to the second binding moiety of the divalent ligand, wherein said first domain is heterologous with respect to said second domain. The first domain of these fusion proteins is the same as the second domain as the chimeric proteins of the invention. Thus, the first domain of said second fusion protein binds the nuclear envelope, the nuclear lamina, heterochromatin, or any combination thereof, and is preferably a nuclear envelope-binding protein, nuclear lamina-binding protein, a heterochromatin-binding protein, the binding moiety of any of those proteins, or any combination thereof.

The second domains of the first and second fusion proteins of this molecular switch system are each capable of specifically binding to one binding moiety of a divalent ligand. The first fusion protein binds to one of the binding moieties (e.g., moiety A) of the divalent ligand and the second fusion protein binds to the other binding moiety (e.g., moiety B) of the divalent ligand. In an embodiment, the second domain of each fusion protein can be a single chain variable region (scFv) of an antibody with specificity for its respective binding moiety of the divalent ligand.

Numerous possibilities exist for moieties A and B. The criteria are that the moiety is sufficiently antigenic to allow selection of a antibody specific for that moiety, and that the two moieties, linked together, form a compound that can enter and act within a cell to mediate formation of the complex. In one embodiment, moiety A can have a structure, for example, as depicted below: 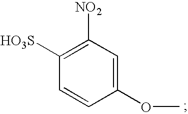
moiety B can have a structure, for example, as depicted below: 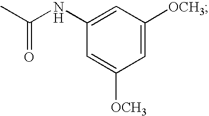
and moieties A and B can be linked by a linker of any suitable length, having units such as those depicted below:

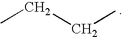

Any compound capable of entry into cell and having moieties against which antibodies can be raised is suitable for the divalent ligand aspect of the invention. This embodiment of the invention permits sequence-specific localization of the target gene domain to the nuclear periphery by allowing a complex to form in the presence of the divalent ligand. In the absence of the divalent chemical no tertiary complex is formed.

In a preferred embodiment, a chemical switch is used which is a divalent chemical comprising two linked compounds. These compounds may be any compounds to which antibodies can be raised linked by a short linker, for example, CH₂CH₂. In one preferred embodiment, a single chain antibody (e.g., a single chain Fᵥ (scFv)) binds to one portion of the divalent chemical to link it to a nucleic acid binding domain. The other portion of the divalent chemical binds to a second single chain antibody, for example a single chain Fᵥ (scFᵥ), which recognizes and binds to protein domain capable of associating with or binding to the nucelar periphery.

In another embodiment, the second domain of the two fusion proteins can be mutant S-tag and S-proteins (described below) which can only bind to each other in the presence of a small molecule or chemical. This small molecule thus acts as the divalent ligand to bring the two fusion proteins into a single complex that localizes to the nuclear periphery and leads to gene repression or down regulation.

This molecular switch system can be used in a method to regulate repression of a target gene in a temporally or spatially manner. In particular the method involves contacting a cell or organism containing a target nucleic acid having a nucleotide sequence associated with a target gene with the molecular switch system of the invention (as described in this section), and contacting the cell or organism with the appropriate divalent ligand at a time or in a location to allow formation of a complex with the fusion proteins and thereby repress or down regulate expression of the target gene by virtue of its localization to the nuclear periphery. As with the chimeric proteins, the fusion proteins of the molecular switch system can be introduced into the cell or organism as proteins, as one or more nucleic acids encoding one or more of the proteins, or as a combination thereof. When a single nucleic acid is used to deliver the fusion proteins, expression of each protein can be coordinately or independently controlled. Likewise the method is useful with the same target genes as contemplated for the methods using the chimeric proteins of the invention.

The fusion proteins can be expressed, isolated and purified as described above for the chimeric proteins. Likewise they can be introduced into the cells or organism as described above for the chimeric proteins.

E. Molecular Switch Systems for Gene Derepression

Molecular switch systems can be provided in another format that allows controlled regulation for derepression of a target gene, i.e., activating expression of a target gene currently being repressed. In this aspect of the invention, the “switch” is used to disrupt the interaction between two fusion proteins (rather than to promote the interaction as in section D). Again, these systems (also called “chemical switches”) provide another tool to manipulate the timing of or location where gene expression is regulated or controlled. Briefly, the molecular switch system introduces two fusion proteins, one with the nucleic acid binding domain and the other with the nuclear periphery-binding domain, into a cell or organism. These two fusion proteins each have second domains which specifically bind to each other, e.g., the second domains are binding partners for one another. In this system, introduction of the fusion proteins leads to formation of a complex which localizes to the nuclear periphery and represses or down regulates expression of the associated target gene. When the chemical switch is introduced into the cells or organisms at the desired time (or in the particular cell types), it acts to disrupt the complex and release the state of repression, i.e., presence of the chemical switch leads to derepression of the target gene.

Accordingly, the first fusion protein of this aspect of the invention comprises a first domain capable of specifically binding a nucleotide sequence associated with a target gene, and a second domain capable of specifically binding to the second binding moiety of the divalent ligand, wherein said first domain is heterologous with respect to said second domain. These fusions proteins are distinct from those described in Section D.

The first domain of the fusion protein is the same as the first domain of the chimeric proteins of the invention. For example, this first domain of the first fusion protein can be a ZFP, an AZP, a leucine zipper protein, a helix-turn-helix protein, a helix-loop-helix protein, a homeobox domain protein, the DNA binding moiety of any of those proteins, or any combination thereof. Likewise, the nucleotide sequence associated with the target gene, and the target gene is the same as described for the chimeric proteins of the invention.

The second fusion protein of this aspect of the invention comprises a first domain capable of associating with the nuclear periphery and a second domain comprising the binding partner of the second domain of said first fusion protein, wherein said first domain is heterologous with respect to said second domain. The first domain of these second fusion proteins is the same as the second domain as the chimeric proteins of the invention. Thus, the first domain of said second fusion protein binds the nuclear envelope, the nuclear lamina, heterochromatin, or any combination thereof, and is preferably a nuclear envelope-binding protein, nuclear lamina-binding protein, a heterochromatin-binding protein, the binding moiety of any of those proteins, or any combination thereof.

The second domains of the first and second fusion proteins of this molecular switch system are each capable of specifically binding to one another. One example of second domains is represented by the S-tag/S-protein system [Kim et al.(1993) Protein Sci. 3:348-356]. The S-tag is a short peptide (15 amino acids) and S-protein is a small protein (104 amino acids) and can be used interchangeably as the second domain for either of the two fusion proteins. The affinity of the S-tag and S-protein complex is high (Kd=1 nM). The chemical switch or ligand is then a molecule which can disrupt the interaction between the S-tag and the S-protein. For example, free or conjugated S-tag protein may act as the chemical switch.

This molecular switch system can be used in a method to regulate repression of a target gene in a temporally or spatially manner. In particular the method involves contacting a cell or organism containing a target nucleic acid having a nucleotide sequence associated with a target gene with a molecular switch system of the invention (as described in this section) and contacting the cell or organism with a ligand at a time or in a location to disrupt association of the first and second fusion proteins and thereby derepress expression of the target gene. As with the chimeric proteins, the fusion proteins of the molecular switch system can be introduced into the cell or organism as proteins, as one or more nucleic acids encoding one or more of the proteins, or as a combination thereof. When a single nucleic acid is used to deliver the fusion proteins, expression of each protein can be coordinately or independently controlled. Likewise the method is useful with the same target genes as contemplated for the methods using the chimeric proteins of the invention.

These fusion proteins can be also be expressed, isolated and purified as described above for the chimeric proteins. Likewise they can be introduced into the cells or organism as described above for the chimeric proteins.

F. Pharmaceutical Formulations

Therapeutic formulations of the chimeric proteins, the molecular switch systems (as provided in Section D or E), the various fusion proteins (of Section D or E) or the nucleic acids encoding any of those proteins or systems of the invention are prepared for storage by mixing those entities having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and can include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

The formulations herein may also contain more than one active compound as necessary for the particular indication being treated, preferably those with complementary activities that do not adversely affect each other. Such molecules are suitably present in combination in amounts that are effective for the purpose intended.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences, 16th edition, Osol, A. Ed. (1980).

The formulations to be used for in vivo administration are sterile. While this can be readily accomplished by filtration through sterile filtration membranes, other sterilization methods can be used provided that the activity of the active ingredients is not destroyed or altered.

Sustained-release preparations may be prepared. Suitable examples of sustained-release preparations include semipermeable matrices of solid hydrophobic polymers containing the polypeptide variant, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

Those of skill in the art can readily determine the amounts of the chimeric proteins, the molecular switch systems (as provided in Section D or E), the various fusion proteins (of Section D or E) or the nucleic acids encoding any of those proteins or systems of the invention to be included in any pharmaceutical composition and the appropriate dosages for the contemplated use.

Throughout this application, various publications, patents, and patent applications have been referred to. The teachings and disclosures of these publications, patents, and patent applications in their entireties are hereby incorporated by reference into this application.

It is to be understood and expected that variations in the principles of invention herein disclosed in exemplary embodiments may be made by one skilled in the art and it is intended that such modifications, changes, and substitutions are to be included within the scope of the present invention.

### EXAMPLE 1

### Repression of Human VEGF-A

To down regulate the expression of the human vascular endothelial growth factor A (VEGF-A) gene, a recombinant construct encoding a chimeric protein (CP1-vegf) containing the 524-amino acid mouse GCL protein [Leatherman et al. (2000)] and an AZP targeted for the sequence 5′-GTG TGG GTG AGT GAG TGT G-3′ (SEQ ID NO: 7) is prepared. A second construct encoding another chimeric protein (CP2-vegf) is prepared using the same mouse GCL protein and an AZP targeted for the sequence 5′-GGG GCT GGG GGC GGT GTC T-3′ (SEQ ID NO: 8). The target nucleotide sequences are from the promoter of the human VEGF-A gene [Tischer et al. (1991) J. Biol. Chem. 266:11947-11954]. The AZPs have 6 zinc fingers, each with the framework sequence of -Pro-Tyr-Lys-Cys-Pro-Glu-Cys-Gly-Lys-Ser-Phe-Ser-Z⁻¹-Ser-Z²-Z³-Leu-Gln-Z⁶-His-Gln-Arg-Thr-His-Thr-Gly-Glu-Lys- (SEQ ID NO: 3); each framework is joined to the next without additional amino acid residues. The identities of the residues that determine DNA binding specificity (Z⁻¹, Z², Z³ and Z6) for CP1-vegf and CP2-vegf are provided in Table 3.

To test for repression activity, the chimeric proteins constructs are co-transfected into the human histiocytic lymphoma cell line U-937 with a luciferase gene reporter plasmid containing the luciferase gene under control of the human VEGF-A native promoter. This luciferase gene reporter plasmid contains nucleotides from −2279 to +1041 of the VEGF-A gene upstream of the luciferase gene [Liu et al. (2001) J. Biol. Chem. 276:11323-11334]. For a positive control, the U-937 cells are transfected with the luciferase gene reporter plasmid alone or co-transfected with the luciferase gene reporter plasmid and a chimeric protein construct (as a protein or as nucleic acid) of GCL and an AZP (or other DNA binding domain) for an unrelated target sequence. A decrease in luciferase activity relative to the control level indicates that CP1-vegf and CP2-vegf down regulate VEGF-A promoter activity.

Alternatively, repression activity can be monitored by treating cells with the CP1-vegf of CP2-vegf proteins or by transfecting the U-937 cells with a nucleic acid encoding the CP1-vegf or CP2-protein, and monitoring the levels of endogenous VEGF-A mRNA by Northern blotting techniques.

TABLE 3

| | Domain/Target | | | | |
|---|---|---|---|---|---|
| Protein | Nucleotides | Z⁻¹ | Z² | Z³ | Z⁶ |
| CP1-vegf | 1 GTGT | Arg | Asn | Ser | Arg |
| | 2 TGGG | Arg | Asp | His | Thr |
| | 3 GTGA | Arg | Thr | Ser | Arg |
| | 4 AGTG | Thr | Asp | His | Gln |
| | 5 GAGT | Arg | Asn | Asn | Arg |
| | 6 TGTG | Thr | Asp | His | Thr |
| CP2-vegf | 1 GGGG | Arg | Asp | His | Arg |
| | 2 GCTG | Thr | Asp | Asp | Arg |
| | 3 GGGG | Arg | Asp | His | Arg |
| | 4 GGCG | Glu | Asp | His | Arg |
| | 5 GGTG | Thr | Asp | His | Arg |
| | 6 GTCT | Glu | Asn | Ser | Arg |

1. A nucleic acid target-specific chimeric protein comprising one or more first domains capable of specifically binding a nucleotide sequence associated with a target gene and one or more second domains capable of associating with the nuclear periphery, wherein at least one of said first domains is heterologous with respect to at least one of said second domains.

2. The chimeric protein of claim 1, wherein said one or more first domains comprise a zinc finger protein (ZFP), an artificial zinc finger protein (AZP), a leucine zipper protein, a helix-turn-helix protein, a helix-loop-helix protein, a homeobox domain protein, the DNA binding moiety of any of said proteins, or any combination thereof.

3. The chimeric protein of claim 2, wherein said AZP comprises at least one zinc finger, said finger, independently, covalently joined to additional fingers, if present, with from 0 to 10 amino acid residues, wherein the amino acids at positions −1, 2, 3 and 6 of the V-helix of the zinc finger are selected as follows:
at position −1, the amino acid is arginine, glutamine, threonine, methionine or glutamic acid;
at position 2, the amino acid is serine, asparagine, threonine or aspartic acid;
at position 3, the amino acid is histidine, asparagine, serine or aspartic acid; and
at position 6, the amino acid is arginine, glutamine, threonine, tyrosine, leucine or glutamic acid.

4. The chimeric protein of claim 2, wherein said AZP comprises at least one zinc finger, each zinc finger independently represented by the formula —X₃-Cys-X₂₋₄-Cys-X₅-Z⁻¹-X-Z²-Z³-X₂-Z⁶-His-X₃₋₅-His-X₄₋, said finger, independently, covalently joined to additional fingers, if present, with from 0 to 10 amino acid residues; wherein
X is, independently, any amino acid and Xₙ represents the number of occurrences of X in the polypeptide chain;
Z⁻¹ is arginine, glutamine, threonine, methionine or glutamic acid;
Z² is serine, asparagine, threonine or aspartic acid;
Z³ is histidine, asparagine, serine, or aspartic acid; and
Z⁶ is arginine, glutamine, threonine, tyrosine, leucine or glutamic acid.

5. The chimeric protein of claim 4, wherein
Z⁻¹ is arginine, glutamine, threonine or glutamic acid;
Z² is serine, asparagine, threonine or aspartic acid;
Z³ is histidine, asparagine, serine or aspartic acid; and
Z⁶ is arginine, glutamine, threonine,or glutamic acid.

6. The chimeric protein of claim 4, wherein the X positions of at least one of said zinc fingers comprise the corresponding amino acids from a Zif268 zinc finger, an Sp1 finger or an Sp1C finger.

7. The chimeric protein of claim 1, wherein said one or more first domains comprise at least three zinc fingers, each zinc finger represented by the formula -Pro-Tyr-Lys-Cys-Pro-Glu-Cys-Gly-Lys-Ser-Phe-Ser-Z-⁻¹-Ser-Z²-Z³-Leu-Gln-Z⁶-His-Gln-Arg-Thr-His-Thr-Gly-Glu-Lys-, said fingers directly joined to one to the other, wherein
Z⁻¹ is arginine, glutamine, threonine, methionine or glutamic acid;
Z² is serine, asparagine, threonine or aspartic acid;
Z³ is histidine, asparagine, serine, or aspartic acid; and
Z⁶ is arginine, glutamine, threonine, tyrosine, leucine, or glutamic acid.

8. The chimeric protein of claim 7, wherein
Z⁻¹ is arginine, glutamine, threonine or glutamic acid;
Z² is serine, asparagine, threonine or aspartic acid;
Z³ is histidine, asparagine, serine or aspartic acid; and
Z⁶ is arginine, glutamine, threonine, or glutamic acid.

9. The chimeric protein of claim 3, wherein said AZP comprises from 3 to 15 zinc fingers, any one or more of which being represented by said formula.

10. The chimeric protein of claim 9, wherein said AZP comprises 7, 8 or 9 zinc fingers.

11. The chimeric protein of claim 10, wherein said AZP comprises 6 zinc fingers.

12. The chimeric protein of claim 1, wherein said one or more second domains directly or indirectly associate with or bind to the nuclear envelope, the nuclear lamina, heterochromatin, or any combination thereof.

13. The chimeric protein of claim 12, wherein one of said second domains is a GCL protein or a binding moiety of a GCL protein.

14. The chimeric protein of claim 12, wherein said one or more second domains comprise a nuclear envelope-binding protein, a nuclear lamina-binding protein, a heterochromatin-binding protein, a protein capable of associating with or binding to any one of the foregoing, the binding moiety of any of said proteins or any combination thereof.

15. The chimeric protein of claim 14, wherein said nuclear lamina-binding protein or the binding moiety of said nuclear lamina-binding protein is a lamin or a lamina-binding protein.

16. The chimeric protein of claim 14, wherein said heterochromatin-binding protein or the binding moiety of said heterochromatin-binding protein is selected from the group consisting of HP1 and a polycomb-group protein.

17. The chimeric protein of claim 1 comprising from one to six first domains and from one to six second domains.

18. The chimeric protein of claim 1 which further comprises a nuclear-localization signal.

19. The chimeric protein of claim 1 which further comprises or a cellular uptake signal.

20. The chimeric protein of claim 19 which further comprises a nuclear-localization signal.

21. A pharmaceutical composition comprising a therapeutically effective amount of the chimeric protein of claim 1 in admixture with a pharmaceutically acceptable carrier.

22. A nucleic acid comprising a nucleotide sequence encoding the chimeric protein of claim 1.

23. An expression vector comprising the nucleic acid of claim 22.

24. A host cell comprising the expression vector of claim 23.

25. A method of preparing a chimeric protein which comprises
(a) culturing the host cell of claim 24 for a time and under conditions to express said chimeric protein; and
(b) recovering said chimeric protein.

26. The expression vector of claim 23, wherein said vector is a eukaryotic expression vector adapted for transfection into a cell containing a target gene for regulation.

27. A pharmaceutical composition comprising a therapeutically effective amount of the nucleic acid or expression vector of claim 22 in admixture with a pharmaceutically acceptable carrier.

28. A method of binding a target nucleic acid with a chimeric protein which comprises contacting a target nucleic acid containing a nucleotide sequence associated with a target gene with the chimeric protein of claim 1 in an amount and for a time sufficient for said protein to bind to said target nucleic acid.

29. A method of repressing or down regulating expression of a target gene which comprises contacting nucleic acid containing a nucleotide sequence associated with or in sufficient proximity to said target gene with a chimeric protein of claim 1 in an amount and for a time sufficient for said chimeric protein to repress or down regulate expression of said target gene.

30. The method of claim 28, wherein said chimeric protein is introduced into a cell or an organism as a protein or as a nucleic acid encoding said protein.

31. The method of claim 28, wherein said chimeric protein further comprises a nuclear-localization signal.

32. The method of claim 28, wherein said chimeric protein further comprises a cellular-uptake signal.

33. The method of claim 28, wherein said target gene encodes a mammalian gene, an insect gene or a yeast gene.

34. The method of claim 33, wherein said target gene is from a mammal and encodes a cytokine, an interleukin, an oncogene, an angiogenesis factor, an anti-angiogenesis factor, a drug resistance protein, a growth factor or a tumor suppressor.

35. The method of claim 28, wherein said target gene encodes a viral gene.

36. The method of claim 35, wherein said viral gene is from a DNA virus.

37. The method of claim 28, wherein said target gene encodes a plant gene.

38. The method of claim 37, wherein said plant gene is from tomato, corn, rice or a cereal plant.

39. The method of claim 28, wherein said target gene is from a commercial animal.

40. A molecular switch system comprising
(a) a first fusion protein comprising a first domain capable of specifically binding a nucleotide sequence associated with a target gene, and a second domain capable of specifically binding to a first binding moiety of a divalent ligand, said ligand capable of uptake by a cell, wherein said first domain is heterologous with respect to said second domain; and
(b) a second fusion comprising a first domain capable of associating with the nuclear periphery and a second domain capable of specifically binding to the second binding moiety of said divalent ligand.

41. The molecular switch system of claim 40, wherein said second domain of each fusion protein is a single chain variable region (scFv) of an antibody with specificity for its respective binding moiety of the divalent ligand.

42. A molecular switch system comprising
(a) a first fusion protein comprising a first domain capable of specifically binding a nucleotide sequence associated with a target gene, and a second domain capable of specifically binding to a binding partner, wherein said first domain is heterologous with respect to said second domain; and
(b) a second fusion protein comprising a first domain capable of associating with the nuclear periphery and a second domain comprising the binding partner of the second domain of said first fusion protein, wherein said first domain is heterologous with respect to said second domain.

43. The molecular switch system of claim 42, wherein said second domain of the first fusion protein is an S-protein and the second domain of said second fusion protein is an S-tag, or vice-a-versa.

44. The molecular switch system of claim 40, wherein the first domain of said first fusion protein comprises a zinc finger protein (ZFP), an artificial zinc finger protein (AZP), a leucine zipper protein, a helix-turn-helix protein, a helix-loop-helix protein, a homeobox domain protein, the DNA binding moiety of any of said proteins, or any combination thereof.

45. The molecular switch system of claim 44, wherein said AZP comprises at least one zinc finger, each zinc finger independently represented by the formula —X₃-Cys-X₂₋₄-Cys-X₅-Z-⁻¹-X-Z²-Z³-X₂-Z⁶-His-X₃₋₅-His-X₄—, said finger, independently, covalently joined to additional fingers, if present, with from 0 to 10 amino acid residues; wherein
X is, independently, any amino acid and Xₙ represents the number of occurrences of X in the polypeptide chain;
Z⁻¹ is arginine, glutamine, threonine, methionine or glutamic acid;
Z² is serine, asparagine, threonine or aspartic acid;
Z³ is histidine, asparagine, serine or aspartic acid; and
Z⁶ is arginine, glutamine, threonine, tyrosine, leucine or glutamic acid.

46. The molecular switch system of claim 45, wherein
Z⁻¹ is arginine, glutamine, threonine or glutamic acid;
Z² is serine, asparagine, threonine or aspartic acid;
Z³ is histidine, asparagine, serine or aspartic acid; and
Z⁶ is arginine, glutamine, threonine or glutamic acid.

47. The molecular switch system of claim 45, wherein the X positions of at least one of said zinc fingers comprise the corresponding amino acids from a Zif268 zinc finger, an Sp1 finger or an Sp1C finger.

48. The molecular switch system of claim 40, wherein the first domain of said first fusion protein comprises at least three zinc fingers, each zinc finger represented by the formula -Pro-Tyr-Lys-Cys-Pro-Glu-Cys-Gly-Lys-Ser-Phe-Ser-Z-⁻¹-Ser-Z²-Z³-Leu-Gln-Z⁶-His-Gln-Arg-Thr-His-Thr-Gly-Glu-Lys-, said fingers directly joined to one to the other, wherein
Z⁻¹ is arginine, glutamine, threonine, methionine or glutamic acid;
Z² is serine, asparagine, threonine or aspartic acid;
Z³ is histidine, asparagine, serine or aspartic acid; and
Z⁶ is arginine, glutamine, threonine, tyrosine, leucine or glutamic acid.

49. The molecular switch system of claim 48, wherein
Z⁻¹ is arginine, glutamine, threonine or glutamic acid;
Z² is serine, asparagine, threonine or aspartic acid;
Z³ is Histidine, asparagine, serine or aspartic acid; and
Z⁶ is arginine, glutamine, threonine or glutamic acid.

50. The molecular switch system of claim 44, wherein said AZP comprises from 3 to 15 zinc fingers, any one or more of which being represented by said formula, or wherein first domain of said first fusion protein comprises from 3 to 15 zinc fingers.

51. The molecular switch system of claim 50, wherein said AZP or said first domain comprises 6, 7, 8 or 9 zinc fingers.

52. The molecular switch system of claim 40, wherein the first domain of said second fusion protein directly or indirectly associates with or binds to the nuclear envelope, the nuclear lamina, heterochromatin, or any combination thereof.

53. The molecular switch system of claim 52, wherein said first domain of said second fusion is a GCL protein or a binding moiety of a GCL protein.

54. The molecular switch system of claim 52, wherein said first domain of said second fusion protein comprises a nuclear envelope-binding protein, a nuclear lamina-binding protein, a heterochromatin-binding protein, a protein capable of associating with or binding to any one of the foregoing, the binding moiety of any of said proteins, or any combination thereof.

55. The molecular switch system of claim 54, wherein said nuclear lamina-binding protein or the binding moiety of said nuclear lamina-binding protein is a lamin or a lamina-binding protein.

56. The molecular switch system of claim 54, wherein said heterochromatin-binding protein or the binding moiety of said heterochromatin-binding protein is selected from the group consisting of HP1 and a polycomb-group protein.

57. A pharmaceutical composition comprising a therapeutically effective amount of the chimeric protein of claim 40 in admixture with a pharmaceutically acceptable carrier.

58. A nucleic acid encoding the first or second fusion protein, or both, of the molecular switch system of.

59. The nucleic acid of claim 58, wherein said first and second fusion proteins are coordinately regulated.

60. The nucleic acid of claim 58, where said first and second fusion proteins are independently regulated.

61. An expression vector comprising the nucleic acid of claim 58.

62. A host cell comprising the expression vector of claim 61.

63. A method of preparing one or more fusion proteins which comprises
(a) culturing the host cell of claim 62 for a time and under conditions to express said one or more fusion proteins; and
(b) recovering said one or more fusion proteins.

64. The expression vector of claim 61, wherein said vector is a eukaryotic expression vector adapted for transfection into a cell containing a target gene for regulation.

65. A pharmaceutical composition comprising a therapeutically effective amount of the expression vector of claim 64 in admixture with a pharmaceutically acceptable carrier.

66. A method of temporally or spatially repressing expression of a target gene which comprises
(a) contacting a cell or an organism containing a target nucleic acid having a nucleotide sequence associated with a target gene with the molecular switch system claim 40, and
(b) contacting said cell or organism with the divalent ligand of said molecular switch system at a time or in a location to allow formation of a complex between said fusion proteins and thereby repress expression of said target gene.

67. A method of temporally or spatially activating gene expression which comprises
(a) contacting a cell or an organism containing a target nucleic acid having a nucleotide sequence associated with a target gene with the molecular switch system of any one of claim 42; and
(b) contacting said cell or organism with a ligand at a time or in a location to disrupt association of the first and second fusion proteins and thereby derepress expression of said target gene.

68. The method of claim 66, wherein the fusion proteins of said molecular switch system are introduced into said cell or organism as proteins, as one or more nucleic acids encoding one or more of said proteins, or as a combination thereof.


SEQUENCE LISTING (21 sequences)

SEQ ID NO: 1 - protein, 25 aa, Artificial
  Zinc finger domain
CXXXXCXXXXXXXXXXXXHXXXXXH

SEQ ID NO: 2 - protein, 32 aa, Artificial
  Second zinc finger domain
XXXCXXXXCXXXXXXXXXXXXHXXXXXHXXXX

SEQ ID NO: 3 - protein, 28 aa, Artificial
  Zinc finger domain
PYKCPECGKSFSXSXXLQXHQRTHTGEK

SEQ ID NO: 4 - protein, 5 aa, Artificial
  Synthetic flexible linker peptide for linking together multi-finger zinc finger domains
GGGGS

SEQ ID NO: 5 - protein, 11 aa, Human immunodeficiency virus
  MISC_FEATURE (1)..(11): HIV Tat protein domain
YGRKKRRQRRR

SEQ ID NO: 6 - DNA, 9 bp, Human immunodeficiency virus
  misc_feature (1)..(9): HIV DNA Binding Domain
gcagaagcc

SEQ ID NO: 7 - DNA, 19 bp, Artificial
  DNA target sequence
gtgtgggtgagtgagtgtg

SEQ ID NO: 8 - DNA, 19 bp, Artificial
  DNA target sequence
ggggctgggggcggtgtct

SEQ ID NO: 9 - protein, 7 aa, Simian virus 40
  MISC_FEATURE (1)..(7): Peptide from SV40 large T antigen
PKKKRKV

SEQ ID NO: 10 - protein, 16 aa, Artificial
  Peptide, residues 43-58 of the Antennapeida homeodomain protein
RQIKIWFQNRRMKWKK

SEQ ID NO: 11 - protein, 34 aa, Herpes Simplex Virus
  MISC_FEATURE (1)..(34): Residues 267-300 of the HSV VP22 protein
DAATATRGRSAASRPTERPRAPARSASRPRRPVE

SEQ ID NO: 12 - protein, 11 aa, Artificial
  Synthetic peptide modeled after the protein transduction domain of the human immunodeficiency virus TAT protein having cellular uptake activity
YARAAARQARA

SEQ ID NO: 13 - protein, 9 aa, Artificial
  Synthetic peptide modeled after the protein transduction domain of the human immunodeficiency virus TAT protein having cellular uptake activity, referred to as “R9”
RRRRRRRRR

SEQ ID NO: 14 - protein, 16 aa, Artificial
  D-penetratin peptide
RQIKIWFQNRRMKWKK

SEQ ID NO: 15 - protein, 16 aa, Artificial
  Peptide Syn B1 from Anntennapedia homeodomain protein
RGGRLSYSRRRFSTST

SEQ ID NO: 16 - protein, 10 aa, Artificial
  L-SynB3 peptide from Antennapedia homeodomain protein
RRLSYSRRRF

SEQ ID NO: 17 - protein, 10 aa, Artificial
  D-SynB3 peptide from Antennapedia homeodomain pro
RRLSYSRRRF

SEQ ID NO: 18 - protein, 8 aa, Artificial
  Flag Epitope Peptide
DYKDDDDK

SEQ ID NO: 19 - protein, 5 aa, Artificial
  Artificial peptide linker
GGGGS

SEQ ID NO: 20 - protein, 4 aa, Artificial
  Artificial peptide linker
GGGS

SEQ ID NO: 21 - protein, 3 aa, Artificial
  Artificial peptide linker
GGS